(19) United States
(12) Patent Application Publication
(10) Pub. No.: US 2025/0235134 A1
(43) Pub. Date: Jul. 24, 2025

(54) Safety Lancet

(71) Applicant: Ryan EL-HOSSEINY, Jupiter, FL (US)

(72) Inventor: Ryan EL-HOSSEINY, Jupiter, FL (US)

(21) Appl. No.: 18/855,871

(22) PCT Filed: Apr. 22, 2022
(86) PCT No.: PCT/US2022/071872

§ 371 Date: Oct. 10, 2024

Related U.S. Application Data
(60) Provisional application No. 63/362,903, filed on Apr. 13, 2022.
(60) Provisional application No. 29/792,642, filed on Apr. 13, 2022.

(51) Int. Cl. A61B 5/15; A61B 5/151

(52) U.S. Cl. CPC A61B 5/150259; A61B 5/150748; A61B 5/150832; A61B 5/15109

(57) ABSTRACT

A safety lancet including a foundation, a base extending upwardly from the foundation, a spring extending upwardly from the base at a first end, the spring connected to a lancet body at a second end of the spring, the lancet body having a lancet extending from a lancet holder, a pair of restriction arms extending from the base, the restriction arms arranged to releasably hold the lancet holder of the lancet body, a housing extending from the base, and a sheath slidably connected to the housing, the sheath having an aperture arranged on an upper surface of the sheath, extending from the distal surface of the sheath are a pair of release means, wherein the pair of release means are arranged to engage the pair of restriction arms when force is imparted on the sheath thereby releasing the spring from a compressed configuration.

### CROSS-REFERENCE TO RELATED APPLICATIONS

This application claims the benefit of and priority to U.S. Design Application No. 29/792,642, filed on Apr. 13, 2022, and entitled “Safety Lancet” and also claims the benefit of U.S. Provisional Application No. 63/362,903, filed on Apr. 13, 2022, and entitled “Safety Lancet”. Both applications are incorporated herein by reference in their entireties.

### FIELD OF THE INVENTION

The present invention relates generally to a safety lancet, specifically the present invention is a lancet needle arranged in a spring-loaded manner.

### BACKGROUND OF THE INVENTION

At-home blood collection has become more commonplace where smaller amounts of blood will satisfy a given diagnostic procedure. Typically, these collections are facilitated by a kit which includes a collection vessel for the blood and a needle-type apparatus for puncturing the skin to provide blood for collection, i.e., a lancet device.

Although safety lancets are well known in the art, prior attempts are configured such that a user needs to bring the lancet device to their finger with their opposite hand while also holding the device in the other hand. Although many healthy people would proceed with the lancing, i.e., puncturing of the skin to draw blood, without a problem—this does not help patients who may have conditions that cause tremors, e.g., multiple sclerosis, post-stroke, traumatic brain injuries, neurodegenerative diseases that affect parts of the brain (e.g., Parkinson's disease), the use of certain medicines (particular asthma medication, amphetamines, caffeine, corticosteroids, and drugs used for certain psychiatric and neurological disorders), alcohol abuse or withdrawal, mercury poisoning, overactive thyroid disorder, liver or kidney failure, anxiety or panic, and the like.

Thus, there is a long felt need for a device that facilitates lancing a person's finger (or foot), where the device is arranged to have a foundation that sturdily supports the device in an upright position and where the foundation includes gripping means to grip the surface under the foundation—eliminating the need for using both hands for lancing.

### SUMMARY OF THE INVENTION

The present invention broadly comprises a safety lancet including a foundation, a base extending upwardly from the foundation, a spring extending upwardly from the base at a first end, the spring connected to a lancet body at a second end of the spring, the lancet body having a lancet extending from a lancet holder, a pair of restriction arms extending from the base, the pair of restriction arms having an end arranged to releasably hold the lancet holder of the lancet body, a housing extending from the base, and a sheath, the sheath slidably connected to the housing, the sheath having an aperture arranged colinearly with the lancet arranged on an upper surface of the sheath, the sheath having a distal surface arranged opposite the upper surface, extending from the distal surface are a pair of release means, wherein the pair of release means are arranged to engage the pair of restriction arms when force is imparted on the sheath thereby releasing the spring from a compressed configuration allowing the lancet body to travel in a direction towards the aperture.

The present invention also broadly comprises a safety lancet having a foundation, a base extending upwardly from the foundation, a lancet fixed secured to a top surface of the base, the lancet extending upwardly therefrom, the lancet having a puncture end arranged opposite of the top surface of the base, a housing extending from the base, a sheath, the sheath slidably connected to the housing, the sheath arranged to slidably surround an outside surface of the housing, the sheath having an aperture arranged colinearly with the lancet arranged on an upper surface of the sheath, and at least one spring secured to said upper surface of said base, wherein the at least one spring forcibly maintains the upper surface of the sheath in a position above the puncture end of the lancet.

The present invention also may comprise a safety lancet having a body, the body having a gripping means arranged on a distal surface, the body having a static portion and a dynamic portion, the dynamic portion slideably secured to the static portion, the dynamic portion having an aperture arranged on an upper surface, a lancet secured to a lancet body, the lancet body in communication with a spring, the spring connected to a distal end of the lancet body and the static portion of the body, a pair of restriction arms extending from the static portion of the body, the pair of restriction arms arranged to secure the lancet body to compress the spring, and a pair of release means extending distally from the dynamic portion of the body, the release means arranged to displace the pair of restriction arms secured on the lancet body thereby releasing the compression of the spring upon receiving a force imparted on the upper surface of the dynamic portion of the body.

The present invention also includes a gripping means, the gripping means arranged on a distal surface of the foundation, where the gripping means may comprise a non-slip rubber, a plurality of protrusions, the plurality of protrusion comprising a non-slip rubber, or releasable gripping means such as hook and loop material that is arranged to engage a hook and loop material on a surface of an objection, or structure, or a suction-cup like structure to secure to a surface of an object, or structure. The gripping means of the foundation provide for sturdy positioning of the present invention during use.

A primary object of the invention is to provide a device that facilitates blood collection without requiring a person to use both hands, that is, the present invention may be positioned in an upright position and requires only one hand to actuate the lancet therein. Specifically, the present invention allows actuation, or access, to a lancet therein by imparting force on a sheath of the present invention.

A secondary object of the invention is to provide a gripping means arranged on a distal surface of a foundation of the invention, where the gripping means imparts an increased coefficient of friction between the invention and an external surface that the invention rests thereon.

These and other objects, features and advantages of the invention will become readily apparent to one having ordinary skill in the art upon study of the following detailed description in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1A illustrates a perspective view of the present invention;

FIG. 1B illustrates a skeleton perspective view of the present invention;

FIG. 2A illustrates a perspective view of the present invention with cap 50 removed;

FIG. 2B illustrates a perspective view of the present invention with lancet 60 exposed after actuation;

FIG. 3A illustrates a perspective view of the present invention with housing 30 removed and sheath 40 partially cut-away;

FIG. 3B illustrates a second perspective view of the present invention with housing 30 removed and sheath 40 partially cut-away;

FIG. 4A illustrates an exploded view of the present invention with housing 30 removed;

FIG. 4B illustrates an exploded view of the present invention with housing 30 removed and sheath 40 partially cut-away;

FIG. 5A illustrates a side view of the present invention with sectional views of housing 30 and sheath 40 taken generally along line 5-5 shown in FIG. 1A;

FIG. 5B illustrates an enlarged view of the section labeled “FIG. 5B” shown in FIG. 5A;

FIG. 6A illustrates a side view of the present invention with sectional views of housing 30 and sheath 40 shown in FIG. 5A, post-actuation;

FIG. 6B illustrates an enlarged view of the section labeled “FIG. 6B” shown in FIG. 6A;

FIGS. 7A-7D illustrate various embodiments of gripping mechanism 11 of foundation 10;

FIG. 8A illustrates a perspective view of an alternative embodiment of the present invention;

FIG. 8B illustrates a skeleton perspective view of the alternative embodiment shown in FIG. 8A;

FIG. 8C illustrates lancet base 20 of the alternative embodiment shown in FIG. 8A;

FIG. 9A illustrates a perspective cut-away view of the alternative embodiment shown in FIG. 8A; and,

FIG. 9B illustrates a perspective cut-away view of the alternative embodiment shown in FIG. 9A, with lancet 60 exposed.

### DETAILED DESCRIPTION OF THE PREFERRED EMBODIMENT

At the outset, it should be appreciated that like reference numbers on different drawing figures represent identical structural elements. It should also be appreciated that, while a number of different embodiments and variations of the present invention are shown in the various drawings, the invention as claimed is not intended to be limited to these specific embodiments, as the claims define a broader invention that can take many different shapes and structures. Also, the adjectives, “top”, “bottom”, “right”, “left”, and their derivatives, in the description herebelow, refer to the perspective of one facing the invention as shown in the figure under discussion.

The term “lancet” should be understood to mean a type of surgical knife with a short, wide, pointed double-edged blade, used especially for making punctures and small incisions. The term “lancet” should also be understood to mean that the lancet possesses a pointed tip, or end, and an oppositely arranged “base”, or end, that is mounted onto a structure. The term “lancing” should be understood to mean a thrusting actuation of a lance to initiate impact with the tip or a motion in the direction towards a lance to initiate impact with the tip. The term “actuation” should be appreciated to mean movement of the present invention, i.e., movement of the sheath and the lancet of the present invention or sole movement of the sheath of the present invention.

It should also be appreciated that the corresponding drawings are merely exemplary illustrations of the present invention, those drawings are not to scale and are provided to example the present invention and its respective components.

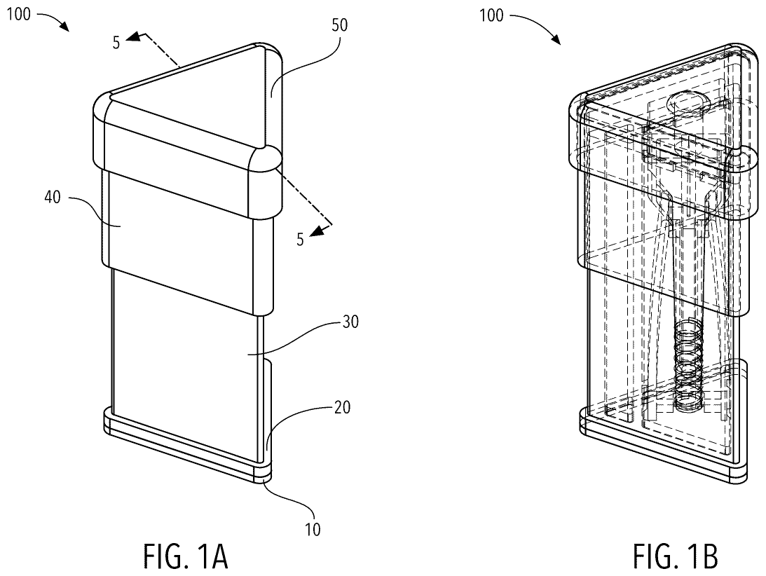

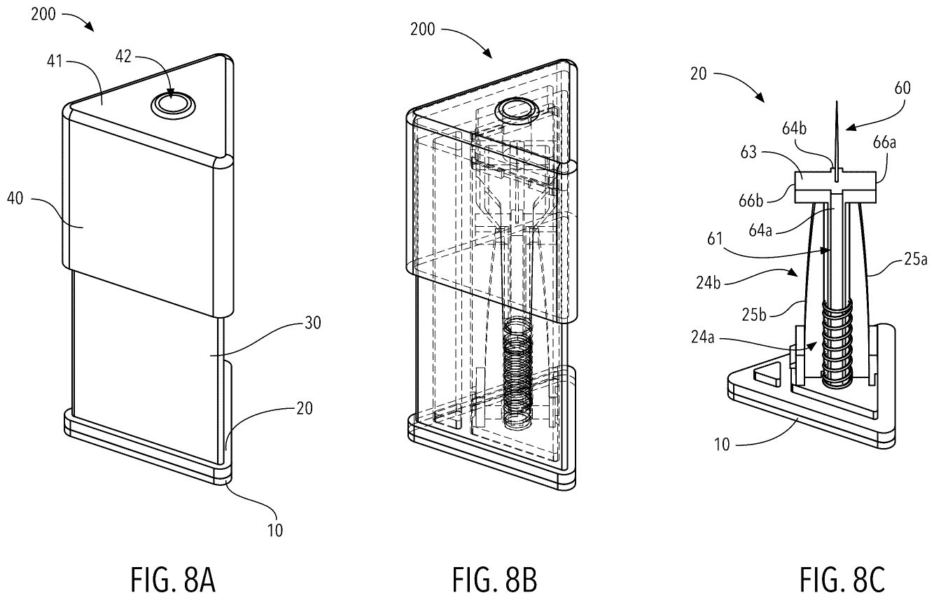

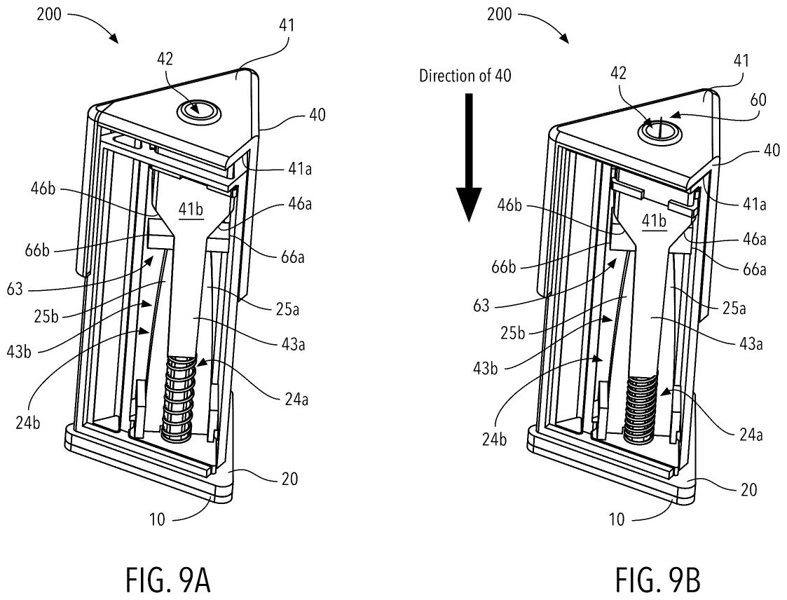

Adverting now to the figures. At the outset, the figures described infra detail a preferred embodiment of the present invention and an alternative embodiment of the present invention. Specifically, FIGS. 1-6B depict the preferred embodiment of the present invention and FIGS. 8A-9B depict an alternative embodiment of the present invention. Safety lancet 100 is the preferred embodiment of the present invention and safety lancet 200 is an alternative embodiment of the present invention. Both safety lancets 100 and 200 include foundation 10 which is specifically arranged to provide for a gripping mechanism of the present invention on a surface underneath to substantially prevent excessive movement of the lancet during actuation. Like components of safety lancets 100 and 200 may share structural components, e.g., lancet base 20, however those like components may have different structural compositions between the embodiments.

The following description should be taken in view of FIGS. 1A and 1B. Safety lancet 100 is the preferred embodiment of the present invention. Safety lancet 100 generally comprises foundation 10, lancet base 20, housing 30, and sheath 40. FIGS. 1A and 1B both illustrate cap 50 which is arranged to cover the top surface of sheath 40. It should also be appreciated that safety lancet 200 (shown in FIGS. 9-13) may also have cap 50. FIG. 1B illustrates a skeleton perspective view of safety lancet 100 to show a generally preferred arrangement of the internal structural components.

Foundation 10 is arranged to have gripping means on its distal surface. Gripping means may include a rubberized construction of the entirety of foundation 10, specifically a non-slip material, such as a non-slip rubber. The gripping means of foundation 10 may embody a plurality of raised protrusions, where each of the protrusions may be comprised of non-slip material. The gripping means of foundation 10 may embody a plurality of ridges, where each of the ridges may be comprised of a non-slip material, or a combination thereof. The gripping means of foundation 10 may include a hook and loop type fastener that is arranged to engage a like hook and loop type fastener that is attached to an external surface. Although foundation 10 is shown to have generally the same external perimeter of lancet base 20, foundation 10 could have an external perimeter that is greater than the external perimeter of lancet base 20. One with ordinary skill in the art would understand that the gripping means of foundation 10 may take a variety of configurations to provide for substantially no translational movement of safety lancet 100 and 200 on an external surface when in use-specifically to allow for a patient to use a single hand without concern for extraneous movement of safety lancets 100 and 200. The material of gripping means of foundation 10 could comprise, anti-slip tapes or threads and could be comprised of a rubber, PVC or polyurethane and may also include a grit surface of a hard substance, such as silicon carbide. Foundation 10 is shown and described in more detail, infra.

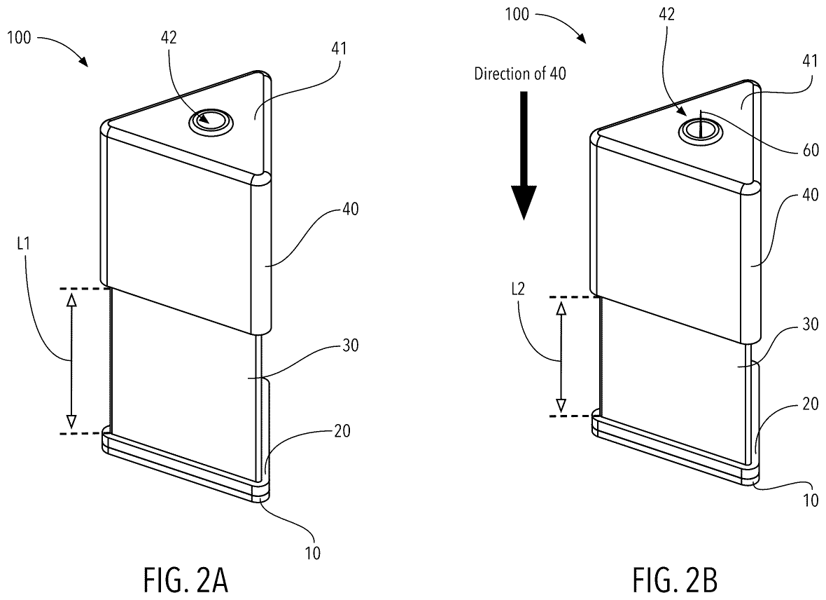

The following description should be taken in view of FIGS. 2A and 2B. FIG. 2A generally illustrates safety lancet 100 prior to actuation of the internal lancet. FIG. 2B generally illustrates safety lancet 100 post-actuation of lancet 60. Lancet aperture 42 is arranged on upper surface 41 of sheath 40. Sheath 40 is slideably attached to housing 30, such that when force is imparted on upper surface 41 of sheath 40, sheath 40 moves in a direction towards foundation 10. When force is imparted on sheath 40, i.e., a downward force on upper surface 41, sheath 40 interacts with lancet base 20 to actuate lancet 60 in a direction towards lancet aperture 42 such that the tip of lancet 60 will be positioned above lancet aperture 42. The actuation of safety lancet 100 is detailed further, infra.

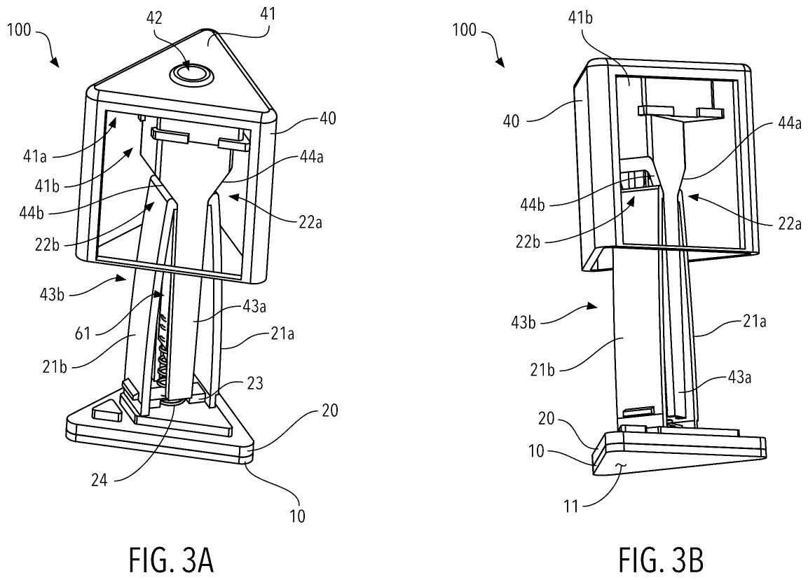

The following description should be taken in view of FIGS. 3A and 3B. FIGS. 3A and 3B both illustrate safety lancet 100 without housing 30 and sheath 40 partially cut-away. First lancet restriction arm 21a, second lancet restriction arm 21b, and spring base 23 all extend from the exposed surface of lancet base 20. Spring 24 is secured to spring base 23 at its distal end and is secured to the distal end of lancet body 61 at its upper end. Constraint end 22a is arranged on first lancet restriction arm 21a, specifically constraint end 22a is arranged distally in relation to lancet base 20. Constraint end 22b is arranged on second lancet restriction 21b, specifically constraint end 22b is arranged distally in relation to lancet base 20. Constraint ends 22a and 22b both collectively hold lancet body 61 in a position that compresses spring 24 preventing movement of lancet body 61.

Channel body 41b extends from distal surface 41a of sheath 40. First channel arm 43a and second channel arm 43b extend distally from channel body 41b. First release means 44a and second release means 44b are arranged on channel body 41b. First release means 44a and second release means 44b of channel body 41b are sloped members and are arranged to interact with constraint end 22a of first lancet restriction arm 21a and constraint end 22b of second lancet restriction arm 21b, to release the compression on spring 24 from constraint ends 22a and 22b. First release means 44a and second release means 44b can take a plurality of forms that would allow their respective interaction with constraint end 22a of first lancet restriction arm 21a and constraint end 22b of second lancet restriction arm 21b to release lancet body 61 therefrom. Specifically, first release means 44a disengages constraint end 22a from lancet body 61 and second release means 44b disengages constraint end 22b from lancet body 61.

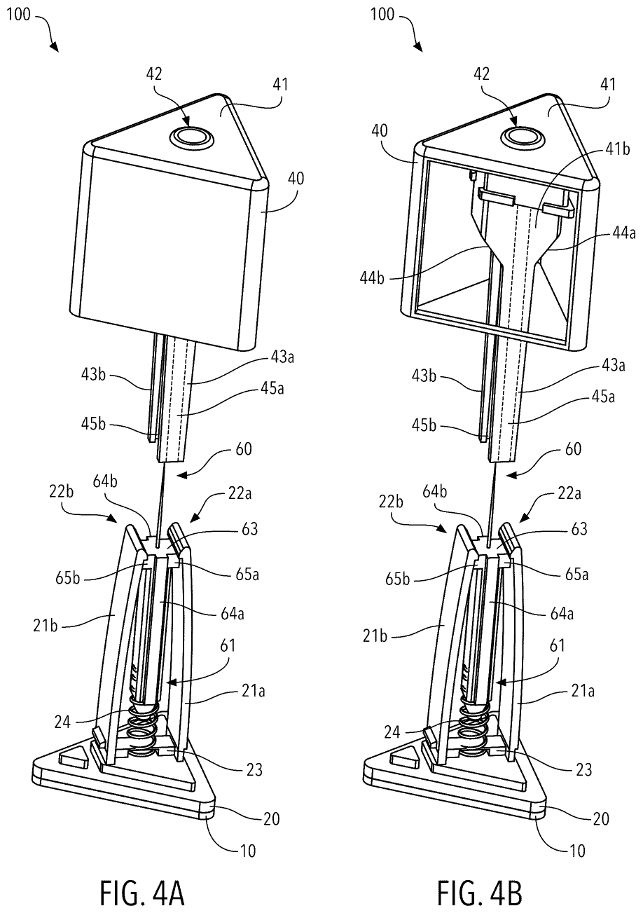

The following description should be taken in view of FIGS. 4A and 4B. FIG. 4A generally illustrates an exploded view of safety lancet 100 without case 30 and FIG. 4B generally illustrates an exploded view of safety lancet 100 without case 30 and with a portion of sheath 40 removed. Lancet body 61 includes spring attachment means 62 and lancet holder 63 which are arranged at opposite ends of lancet body 61. Spring attachment means 62 may comprise a plurality of configurations to fixedly secure spring 24 thereto, such as threadable means, an aperture arranged to accept spring 24 therein, or the like. Lancet holder 63 is primarily arranged to function as a base for the non-tipped end of lancet 60 to be fixed secured thereto. Lancet holder 63 includes first restriction means 65a and second restriction means 65b extending therefrom. First restriction means 65a and second restriction means 65b are essentially ridges, or shelves, that are arranged to provide a surface for the respective constraint ends 22a and 22b to hold lancet body 61 in a position to compress spring 24. First guide means 64a and second guide means 64b are disposed on opposite external surfaces of lancet body 61. First guide means 64a and second guide means 64b are ridges, or like structures, that are arranged to slide within channel 45a of first channel arm 43a and channel 45b of second channel arm 43b, respectively (first channel 45a is illustrated in hidden lines). First guide means 64a and second guide means 64b work collectively with channel 45a of first channel arm 43a and channel 45b of second channel arm 43b to ensure that lancet body 61 travels in a substantially colinearly vertical direction towards a center point of aperture 42 when the compression imparted on spring 24 is released.

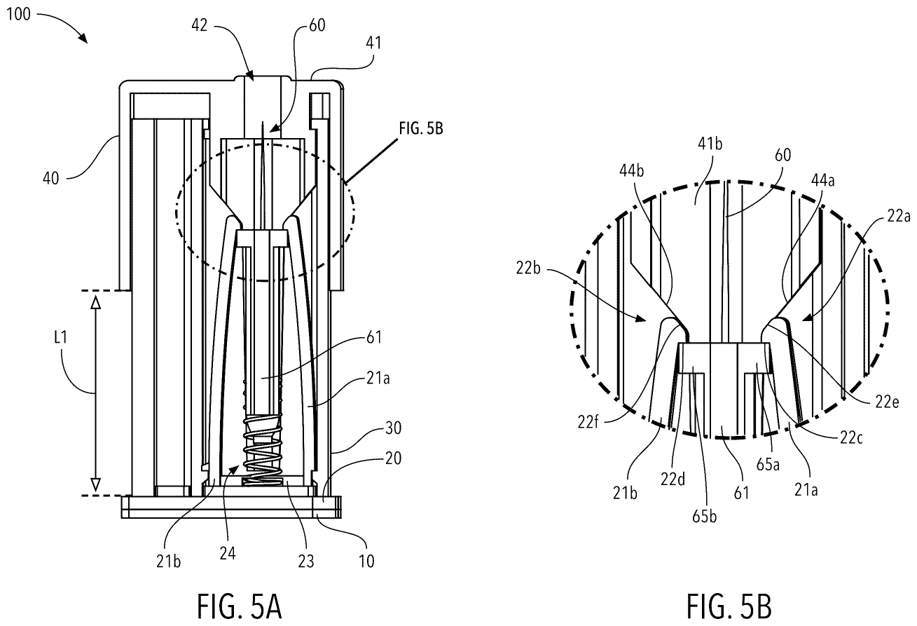

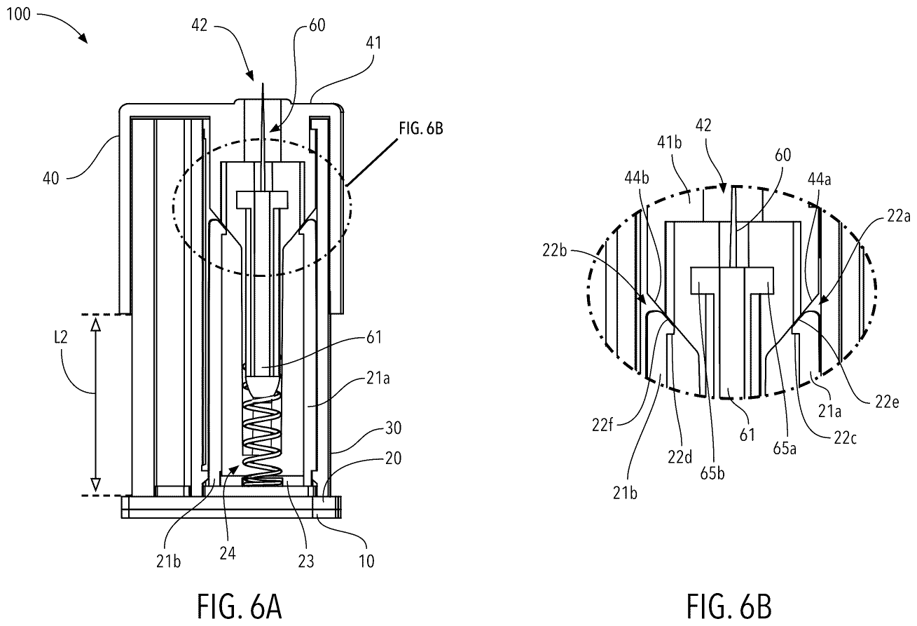

The following description should be taken in view of FIGS. 5A-6B, which illustrate the release configuration of safety lancet 100 when in use. Specifically, FIG. 5A illustrates a partial cross-sectional view of safety lancet 100 where housing 30 and sheath 40 are cross-sectionals taken generally along line 5-5 in FIG. 1A. FIG. 5B illustrates an enlarged view taken from FIG. 5A. FIGS. 5A and 5B illustrate safety lancet 100 prior to actuation of lancet 60. FIG. 6A illustrates a partial cross-sectional view of safety lancet 100 where housing 30 and sheath 40 are cross-sectionals generally shown in FIG. 5A where lancet 60 has been actuated from force imparted on sheath 40. FIG. 6B illustrates an enlarged view taken from FIG. 6A.

As described above, lancet body 61 is secured in a position that compresses spring 24 via constraint end 22a of first lancet restriction arm 21a and constraint end 22b of second lancet restriction arm 21b. Constraint ends 22a and 22b hold first restriction means 65a and second restriction means 65b of lancet body 61, respectively. Constraint ends 22a and 22b have hooks 22c and 22d, respectively, which are edges, shelves, or ridges, that are arranged to abut that protruding configuration of first restriction means 65a and second restriction means 65b of lancet body 61. Hooks 22c and 22d of constraint ends 22a and 22b keep lancet body 61 in a position that maintains compression on spring 24.

To release the compression imparted on spring 24 from lancet body 61, hooks 22c and 22d of constraint ends 22a and 22b are released by first and second release means 44a and 44b of channel body 41b of sheath 40 when sheath 40 is pushed in a downward direction on housing 30, i.e., exposed length L1 of housing 30 transitions to exposed length L2 of housing 30 when sheath 40 is forcibly slid along the outside of housing 30. First and second release means 44a and 44b of channel body 41b are preferably sloped members that are arranged to interact with tapered tips 22e and 22f of constraint ends 22a and 22b, respectively. When first and second release means 44a and 44b are pushed into tapered tips 22e and 22f of constraint ends 22a and 22b, the force thereon displaces hooks 22c and 22d of constraint ends 22a and 22b from first restriction means 65a and second restriction means 65b of lancet body 61, thereby releasing the compression from spring 24. Specifically, tapered tips 22e and 22f of constraint ends 22a and 22b travel along the surface of first and second release means 44a and 44b as sheath 40 is pushed downwardly until hooks 22c and 22d are displaced from first restriction means 65a and second restriction means 65b of lancet body 61, respectively. When the compression of spring 24 is released, lancet body 61 and lancet 60 travel in the direction towards upper surface 41 of sheath 40, up and through lancet aperture 42, exposing the sharp tip of lancet 60 above the upper surface of lancet aperture 42.

It should be appreciated that only a downward force imparted on upper surface 41 of sheath 40 (or the upper surface of lancet aperture 42 arranged on upper surface 41 of sheath 40) is needed to actuate lancet 60 through lancet aperture 42. Foundation 10, and its respective gripping mechanisms (described infra) keep safety lancet 100 in a substantially upright position during actuation, such that a patient does not need to use their opposite hand to facilitate actuation of safety lancet 100.

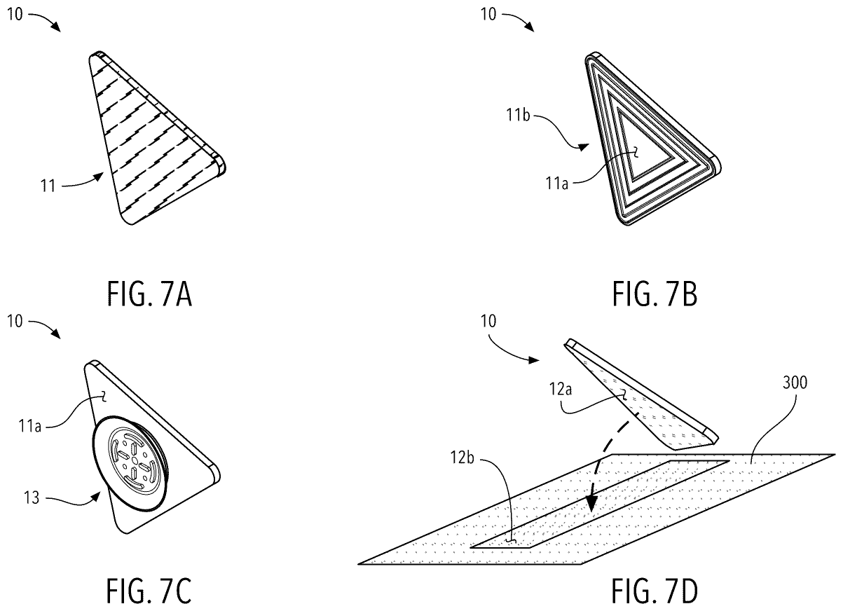

Adverting now to FIGS. 7A-7D which illustrate gripping mechanism 11 of foundation 10 which are configured on both safety lancets 100 and 200. As described supra and shown in FIG. 7A, gripping means 11 of foundation 10 may be a rubberized construction of the entirety of foundation 10, where the rubber is preferably constructed from a non-slip material, or other alternative materials with non-slip compositions instead of rubber. Alternatively, as shown in FIG. 7B, gripping means 11 may be comprised of plurality of ridges 11b that extend from distal surface 11a of foundation 10, where plurality of ridges 11b and distal surface 11a may be both comprised of non-slip rubber, or similar material. Further, as shown in FIG. 7C, gripping means 11 may comprise suction cup 13 extending from distal surface 11a of foundation 10, where suction cup 13 is arranged to removably secure safety lancets 100 and 200 on an external surface. Further still, as shown in FIG. 7D, gripping means 11 may include hook and loop fastener 12a which is arranged to secure to hook and loop fastener 12b arranged on external surface 300, to removably secure safety lancets 100 and 200, thereto. It should be noted that hook and loop fasteners 12a and 12b may comprise any interference-based type of removable connection means.

Gripping means 11, illustrated in FIGS. 7C and 7D, may be more preferable in clinical settings where it is more advantageous to secure safety lancets 100 and 200 to a predetermined area.

It should be appreciated that gripping means 11 of foundation 10 increases the coefficient of friction between an external surface that gripping means 11 of foundation 10 contacts when safety lancets 100 and 200 are resting on the external surface. Coefficient of friction is understood to mean the ratio of the frictional force resisting the motion of two surfaces in contact to the normal force pressing the two surfaces together. Coefficient of friction is symbolized by mu (μ) and follows the formula provided below, where F is the frictional force and N is the normal force:

$$μ = F / N$$

A low value of coefficient of friction (μ) indicates that the force required for sliding to occur is less than the force required when the coefficient of friction (μ) is high. If safety lancets 100 and 200 did not include gripping means 11, the coefficient of friction would be represented by μ₁. When safety lancets 100 and 200 include gripping means 11, the coefficient of friction is represented by μ₂. Thusly, gripping means 11 results in a relationship of:

$${μ}_{2} > {μ}_{1}$$

That is, the coefficient of friction between safety lancets 100 and 200 having gripping means 11 and an external surface is greater than the coefficient of friction between safety lancets 100 and 200 not having gripping means and an external surface. This increase in the coefficient of friction afforded by gripping means 11 of safety lancets 100 and 200 helps to prevent either of the safety lancets from moving should a patient have a condition that cause tremors which could make the lancing process, i.e., actuation of lancet 60, more difficult, e.g., safety lancets 100 and/or 200 falling over during the lancing process from patient tremors.

Adverting now to the alternative embodiment of the present invention, safety lancet 200 is shown in FIGS. 8A-9B. It should be appreciated that many of the components of safety lancet 100, described supra, are shared with safety lancet 200. However, lancet base 20 of safety lancet 200 and channel body 41b of safety lancet 200 comprise a different configuration than that of safety lancet 100.

The following description should be taken in view of FIGS. 8A-8C. Safety lancet 200 generally comprises foundation 10, lancet base 20, housing 30, and sheath 40. As shown in FIG. 9B, lancet base 20, housing 30, and sheath 40 collectively house and hold the internal components of safety lancet 200.

Unlike lancet base 20 of safety lancet 100, lancet base 20 of safety lancet 200 fixes lancet body 61 to lancet base 20. Lancet body 61 is buttressed by first support 25a and second support 25b. Lancet body 61, first support 25a and second support 25b may be integral with lancet base 20 of safety lancet 200. Lancet base 20 of safety lancet 200 includes two springs, 24a and 24b that are arranged to interact with sheath 40. Specifically, springs 24a and 24b impart force onto first channel arm 43a and second channel 43b to keep sheath 40 in a position where the tip of lancet 60 is within sheath 40 as shown in FIG. 9A, i.e., lancet 60 not exposed through aperture 42 as shown in FIG. 9B.

Lancet body 61 of safety lancet 200 does not include the first and second restriction means 65a and 65b like safety lancet 100 (shown in FIG. 5B) but rather includes first and second restriction means 66a and 66b which are arranged to interact with sheath 40. Specifically, first and second restriction means 66a and 66b of lancet body 61 of safety lancet 200 are arranged to prevent sheath 40 from traveling in a downward direction past first and second restriction means 66a and 66b.

The following description should be taken in view of FIGS. 9A and 9B. FIG. 9A illustrates a perspective view of safety lancet 200 with portions of housing 30 and sheath 40 removed. FIG. 9B illustrates a perspective view of safety lancet 200 with lancet 60 exposed along with portions of housing 30 and sheath 40 removed. Like safety lancet 100, sheath 40 is slideably attached to housing 30 of safety lancet 200. In a resting position, lancet 60 is disposed within sheath 40 of safety lancet 20. During actuation of safety lancet 200, force is imparted on upper surface 41 of sheath 40 to slide sheath 40 in a downward direction on housing 30 towards foundation 10, thereby exposing lancet 60 above the top surface of aperture 40 and through aperture 42.

Channel body 41b of safety lancet 200 includes channel 45a within first channel arm 43a and channel 45b of second channel arm 43b (not shown but identical to safety lancet 100 shown in FIGS. 4A and 4B). Like safety lancet 100, channel 45a within first channel arm 43a and channel 45b of second channel arm 43b are arranged to accept first guide means 64a and second guide means 64b, respectively, of safety lancet 200 to provide a movement guide of sheath 40. Channel body 41b of safety lancet 200 also includes first limiting means 46a and second limiting means 46b. First limiting means 46a and second limiting means 46b are arranged to contact first and second restriction means 66a and 66b of lancet body 20 to prevent sheath 40 from traveling in a downward direction past first and second restriction means 66a and 66b of safety lancet 200.

The notable difference between safety lancet 100 and safety lancet 200 is the movement of lancet 60, where lancet 60 of safety lancet 100 is arranged to move in an upward direction towards aperture 42 when force is imparted onto sheath 40 and inversely, where lancet 60 is fixed secured to lancet base 20 in safety lancet 200 and is only exposed via a downward force imparted on sheath 40 to expose lancet 60 as shown in FIG. 9B. It can be said that safety lancet 100 has a configuration that affords a dynamic (moveable) lancet and that safety lancet 200 has a configuration where the lancet is in a static (fixed) position. Safety lancet 200 notably has fewer movable components than safety lancet 100.

The following description should be taken in view of the aforementioned Figures. Foundation 10 and its gripping means 11, housing 30, lancet base 20 are also known as the static portion of the body of safety lancet 100. Sheath 40 is also known as the dynamic portion of the body of safety lancet 100, where the body of safety lancet 100 comprises both the static portion and the dynamic portion. Foundation 10 and its gripping means 11, housing 30, lancet base 20 are also known as the static portion of the body of safety lancet 200. Sheath 40 is also known as the dynamic portion of the body of safety lancet 200, where the body of safety lancet 200 comprises both the static portion and the dynamic portion.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

### REFERENCE NUMERALS

10 Foundation
11 Gripping means of foundation 10
11a Distal surface of foundation 10
12a Hook and loop fastener
12b Hook and loop fastener
13 Suction cup
20 Lancet base
21a First lancet restriction arm of lancet base 20
21b Second lancet restriction arm of lancet base 20
22a Constraint end of first lancet restriction arm 21a
22b Constraint end of second lancet restriction arm 21b
22c Hook of constraint end 22a
22d Hook of constraint end 22b
22e Tapered tip of constraint end 22a
22f Taper tip of constraint end 22b
23 Spring base of lancet base 20
24 Spring
24a First spring
24b Second spring
25a First support of lancet base 20
25b Second support of lancet base 20
30 Housing
40 Sheath
41 Upper surface of sheath 40
41a Distal surface of sheath 40
41b Channel body of sheath 40
42 Lancet aperture of sheath 40
43a First channel arm of channel body 41b
43b Second channel arm of channel body 41b
44a First release means of channel body 41b
44b Second release means of channel body 41b
45a Channel of first channel arm 43a
45b Channel of second channel arm 43b
46a First limiting means
46b Second limiting means
50 Cap
60 Lancet
61 Lancet body
62 Spring attachment means of lancet body 61
63 Lancet holder of lancet body 61
64a First guide means of lancet body 61
64b Second guide means of lancet body 61
65a First restriction means of lancet body 61
65b Second restriction means of lancet body 61
66a First restriction means of lancet body 61
66b Second restriction means of lancet body 61
100 Safety lancet
200 Alternative safety lancet
300 External surface
L1 Exposed length of housing 30
L2 Exposed length of housing 30

1. A safety lancet, comprising:
a foundation;
a base extending upwardly from said foundation;
a spring having a first end and a second end, said spring extending upwardly from said base at said first end of said spring, said spring connected to a lancet body at said second end of said spring, said lancet body having a lancet extending from a lancet holder;
a pair of restriction arms extending from said base, said pair of restriction arms each having an end arranged to releasably hold said lancet holder of said lancet body;
a housing extending from said base; and,
a sheath, said sheath slidably connected to said housing, said sheath having an aperture arranged colinearly with said lancet arranged on an upper surface of said sheath, said sheath having a distal surface arranged opposite said upper surface, extending from said distal surface are a pair of release means,
wherein said pair of release means are arranged to engage each end of said pair of restriction arms when force is imparted on said sheath thereby releasing said spring from a compressed configuration allowing said lancet body to travel in a direction towards said aperture.

2. The safety lancet recited in claim 1, wherein said foundation includes a gripping means, said gripping means arranged on a distal surface of said foundation.

3. The safety lancet recited in claim 2, wherein said gripping means comprises a non-slip rubber.

4. The safety lancet recited in claim 2, wherein said gripping means comprises a plurality of protrusions, said plurality of protrusion comprising a non-slip material.

5. The safety lancet recited in claim 2, wherein said gripping means comprises a plurality of ridges, said plurality of ridges comprising a non-slip material.

6. The safety lancet recited in claim 1, wherein said lancet body includes a pair of guide means, said guide means are arranged to slide within a pair of channels arranged on said pair of release means.

7. The safety lancet recited in claim 1 further comprising a removable cap arranged to cover said upper surface of said sheath.

8. A safety lancet, comprising:
a foundation;
a base extending upwardly from said foundation;
a lancet fixedly secured to a top surface of said base, said lancet extending upwardly therefrom, said lancet having a puncture end arranged opposite of said top surface of said base;
a housing extending from said base;
a sheath, said sheath slidably connected to said housing, said sheath arranged to slidably surround an outside surface of said base, said sheath having an aperture arranged colinearly with said lancet arranged on an upper surface of said sheath; and,
at least one spring secured said upper surface of said base,
wherein said at least one spring forcibly maintains said upper surface of said sheath in a position above said puncture end of said lancet.

9. The safety lancet recited in claim 8, wherein said foundation includes a gripping means, said gripping means arranged on a distal surface of said foundation.

10. The safety lancet recited in claim 9, wherein said gripping means comprises a non-slip rubber.

11. The safety lancet recited in claim 9, wherein said gripping means comprises a plurality of protrusions, said plurality of protrusion comprising a non-slip rubber.

12. The safety lancet recited in claim 8, wherein said lancet is fixedly secured to lancet body, said lancet body extending from said base, wherein said lancet body includes at least one restriction means.

13. The safety lancet recited in claim 12, wherein said sheath includes a distal surface arranged opposite said upper surface, extending from said distal surface is at least one limiting means, wherein said at least one limiting means is arranged to abut said restriction means of said lancet body when said sheath is compressed by said sheath thereby limiting slidably translational movement of said sheath.

14. The safety lancet recited in claim 8 further comprising a removable cap arranged to cover said upper surface of said sheath.

15. The safety lancet recited in claim 9, wherein said gripping means comprises a plurality of ridges, said plurality of ridges comprising a non-slip material.

16. A safety lancet, comprising:
a body, said body having a gripping means arranged on a distal surface, said body having a static portion and a dynamic portion, said dynamic portion slideably secured to said static portion, said dynamic portion having an aperture arranged on an upper surface;
a lancet secured to a lancet body, said lancet body in communication with a spring, said spring connected to a distal end of said lancet body and said static portion of said body;
a pair of restriction arms extending from said static portion of said body, said pair of restriction arms arranged to secure said lancet body to compress said spring; and,
a pair of release means extending distally from said dynamic portion of said body, said release means arranged to displace said pair of restriction arms secured on said lancet body thereby releasing said compression of said spring upon receiving a force imparted on said upper surface of said dynamic portion of said body.

17. The safety lancet recited in claim 16, wherein said lancet body includes a pair of guide means, said guide means are arranged to slide within a pair of channels arranged on said pair of release means.

18. The safety lancet recited in claim 16, wherein said gripping means comprises a non-slip rubber.

19. The safety lancet recited in claim 16, wherein said gripping means comprises a suction cup.

20. The safety lancet recited in claim 16, wherein said gripping means imparts an increased coefficient of friction between said safety lancet and an external surface.
